(19) United States
(12) Patent Application Publication
(10) Pub. No.: US 2009/0095300 A1
(43) Pub. Date: Apr. 16, 2009

(54) Breathing Apparatus

(75) Inventor: Roger McMorrow, Portobello (IE)

Correspondence Address: CROWELL & MORING LLP;INTELLECTUAL PROPERTY GROUP, P.O. BOX 14300, WASHINGTON, DC, 20044-4300, US

(73) Assignee: Roger McMorrow, Dublin, Portobello (IE)

(21) Appl. No.: 12/280,445

(22) PCT Filed: Feb. 23, 2007
(86) PCT No.: PCT/EP2007/001583

§ 371 Date: Dec. 5, 2008

(30) Foreign Application Priority Data
Feb. 24, 2006 (GB) 0603725.3

(51) Int. Cl. A61M 16/22; A61M 16/00; A61M 16/08

(52) U.S. Cl. 128/205.12; 128/204.18; 128/205.17

(57) ABSTRACT

A breathing apparatus (10) comprises a breathing circuit which includes a gas reservoir (50) having one end (54) open to the atmosphere. The reservoir comprises a substantially rigid structure and may be supplemented by an inflatable bag. The circuit is connected, in use, to an oxygen supply container (60), the apparatus further including means (62) for regulating the flow of oxygen into the circuit in response to detect gas levels in the circuit. The preferred apparatus is portable and provides an efficient use of oxygen.

### FIELD OF THE INVENTION

The present invention relates to breathing apparatus.

### BACKGROUND TO THE INVENTION

There are two types of conventional breathing apparatus: open circuit and closed circuit. With an open circuit breathing apparatus, a user inhales a breathable mix of gas from a single or plurality of storage cylinders and exhales into the atmosphere. With a closed circuit breathing apparatus, the user inhales a breathable mix of gas partly from a storage cylinder (or plurality of storage cylinders) and partly from a store of the user's previously exhaled gas. Thus in a closed circuit the user's exhaled gas is recycled.

Recycling the user's exhaled gas, i.e. rebreathing unused oxygen, reduces oxygen wastage and may reduce the amount of breathable gas required to be carried by the user in storage cylinders. Thus a closed circuit breathing apparatus is more efficient and in certain circumstances may be more portable.

In conventional closed circuit breathing apparatus, the exhaled gas is recycled by first reducing the level of unwanted gases, such as carbon dioxide, and then storing the exhaled gas in a sealed reservoir such as a flexible bag. Problems that may occur with conventional closed circuit breathing apparatus include that the reservoir may rip if the volume of exhaled gas is greater that the capacity of the reservoir, that the airway pressure may rise to dangerous levels requiring a pressure reducing valve, that the reservoir may hold an insufficient volume of gas for the user to inhale adequately, and that the reservoir may freeze at low temperatures resulting in the apparatus failing.

It would be desirable to provide an apparatus that mitigates the problems identified above.

### SUMMARY OF THE INVENTION

Accordingly, the invention provides a breathing apparatus comprising a breathing circuit into which gas may be exhaled by a user and from which gas may be inhaled by the user, the circuit including a gas reservoir into which at least some of the exhaled gas is directed and from which at least some of the inhaled gas is drawn, wherein a portion of the gas reservoir is in fluid communication with an external environment, and wherein the circuit is connected, in use, to an apparatus for supplying breathable gas.

The reservoir may be open to the environment by means of one or more apertures and/or by forming a portion of the reservoir from a gas permeable material.

Advantageously, the apparatus includes means for absorbing or removing gas from the exhaled gas. Preferably, the absorbing means is arranged in said breathing circuit such that said exhaled gas passes through said absorbing means before entering said reservoir. Typically, the absorbing means comprises a carbon dioxide absorber.

Typically, the apparatus includes, or is adapted to receive, a container for storing a breathable gas, typically oxygen, most typically pressurised oxygen, the storage device being connectable to the breathing circuit in order to allow a quantity of said breathable gas to be introduced into the breathing circuit, conveniently into said inhalation gas.

In a preferred arrangement, the reservoir includes a first inlet for said exhaled gas and a second inlet or port for allowing gas to be drawn into the reservoir from the external environment, said first and second inlets being distally located in the reservoir. Preferably, the reservoir further includes an outlet by which gas may be drawn from the reservoir for inhalation by the user, said outlet being located proximal said first inlet.

Preferably, the volume of the reservoir is at least equal to the user's maximum tidal volume (typically at least 4 litres), preferably at least twice the user's tidal volume. In the preferred embodiment, the volume of the reservoir is approximately 12 litres.

The reservoir is preferably rigid or semi-rigid, or is at least self-supporting such that it does not collapse during normal use.

In a particularly preferred embodiment, the breathing apparatus comprises a mask, the mask forming a compartment over the user's mouth and/or nose during use, attachable to the mask being means for directing exhaled gas from the mask to the gas absorber, attachable to the gas absorber being said reservoir, further attachable to reservoir being means for directing gas from the reservoir to the mask, a non-exhaled gas supply, said non-exhaled gas supply typically comprising a variable regulator, and means for directing the non-exhaled gas supply the mask (directly or indirectly), the reservoir being substantially non-collapsible, being dimensioned to hold equal to or more than the volume of exhaled gas of the user, and being partially open to, or in fluid communication with, the atmosphere.

The means of reducing the carbon dioxide content of the gas may be a chamber comprising a carbon dioxide absorber or scrubber. A variety of means of absorbing carbon dioxide may be used, for example lithium hydroxide, sodium hydroxide or calcium hydroxide. The chamber may also preferably comprise a tap to drain water which results from the reaction of carbon dioxide with the absorber and also condensation from the users exhaled gas.

In a more preferred embodiment, the apparatus may comprise electronic monitors. An electronic oxygen monitor for example, may monitor the level of oxygen in the reservoir and automatically adjust the amount of gas released from the storage cylinder. A carbon dioxide monitor may be provided for monitoring the build up of carbon dioxide in the circuit and for adjusting the flow of gas to prevent dangerously high levels of carbon dioxide from building up.

The preferred semi-closed circuit breathing apparatus may be used to provide any level of breathable gas, in particular oxygen representing from 0 to almost 100% of the available atmosphere, in any environment: the apparatus may provide a breathable gas in environments where the natural atmosphere is unsuitable, for example at high altitudes where the oxygen level is low, or simulate a low oxygen level at sea level. The apparatus may therefore be used during sports such as mountaineering and for training, especially hypoxic training at sea level. The apparatus may also be used for medical purposes such as to provide breathable gas during sedation or anaesthesia. The portability of the apparatus is particularly beneficial thus the apparatus may be used for example in ambulances, patient transport, weaning from oxygen therapy, ambulatory oxygen therapy, or disaster relief. By providing a suitable filter, for example a nuclear, biological and/or chemical filter, at or adjacent the open port of the reservoir, the apparatus could be used in otherwise dangerous environments. In preferred modes of use, the apparatus acts as a supplementary oxygen device for providing the wearer with levels of oxygen that are higher, or at least different, than can be obtained from the atmosphere.

The advantages of the present invention include that it provides a more efficient use of a stored gas supply, that a reduced amount of breathable gas may be carried by the user in storage cylinders, thus the semi-closed circuit breathing apparatus may be more portable, and that to provide a controlled gas mixture there is no need to carry gas cylinders containing atmospheric gases other than oxygen as the proportion of the required second gas, in particular nitrogen, which would replace oxygen when decreasing the oxygen level of the breathable gas.

The oxygen source need not be from a pressurised container—it may be from other sources, for example a source of chemically stored oxygen, or chilled liquid oxygen or from a piped oxygen supply.

Additional gases, for example helium, may be added to the circuit to reduce the viscosity and density of the circulating gas to ease the work involved in breathing.

Further advantageous aspects of the invention will become apparent to those ordinarily skilled in the art upon review of the following description of a specific embodiment and with reference to the accompanying drawings.

### BRIEF DESCRIPTION OF THE DRAWINGS

Embodiments of the invention is now described by way of example and with reference to the accompanying drawings in which like numerals are used to indicate like parts and in which:

FIG. 1A shows a perspective view of a semi-closed circuit breathing apparatus embodying the invention;

FIG. 1B shows a front view of the semi-closed circuit breathing apparatus of FIG. 1A;

FIG. 1C shows a side view of a carbon dioxide absorber and a reservoir included in the apparatus of FIGS. 1A and 1B;

FIG. 2A shows a front view of a semi-closed circuit breathing apparatus being an alternative embodiment of the invention;

FIG. 2B shows a side view of a carbon dioxide absorber, a reservoir and a storage cylinder included in the apparatus of FIG. 2A;

FIGS. 3A to 3E show respectively a front view, a plan view, a bottom view and side views of the carbon dioxide absorber and reservoir, shown without a cylinder holding portion;

FIGS. 4A to 4C show respectively a plan view and side views of a cylinder holding portion of the apparatus of FIG. 2A; and

FIGS. 5A to 5C show respectively in schematic form alternative breathing circuits suitable for use in apparatus embodying the invention.

### DETAILED DESCRIPTION OF THE DRAWINGS

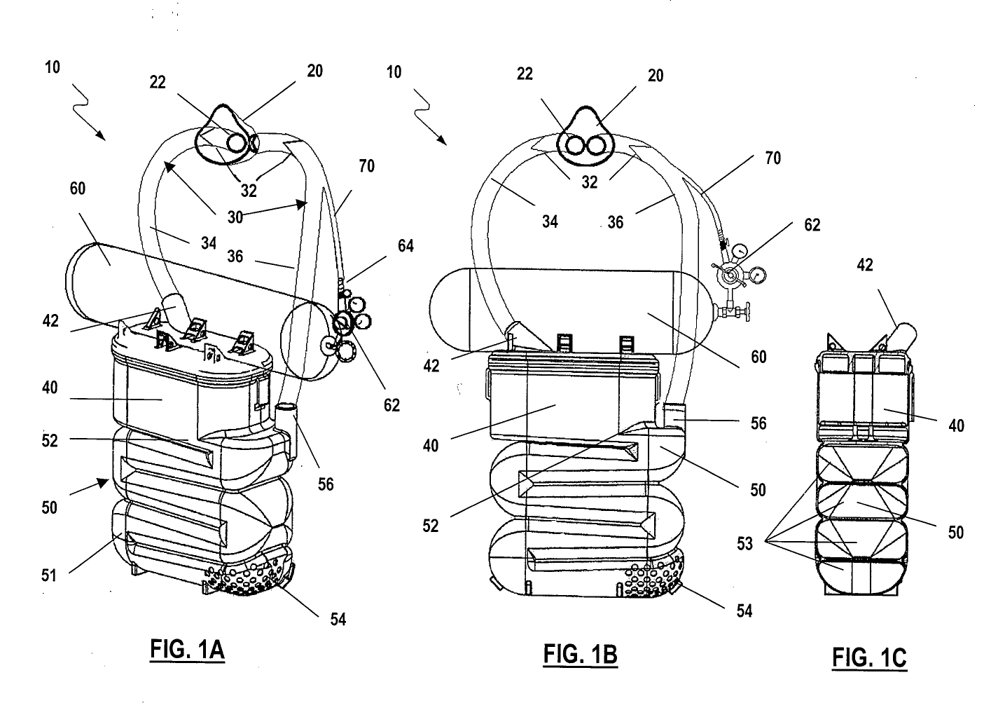

Referring now to FIG. 1A of the drawings, there is shown, generally indicated as 10, a semi-closed circuit breathing apparatus embodying the invention.

The breathing apparatus 10 comprises a mask 20, adapted to fit over a user's mouth and/or nose (not shown). Typically, the mask 20 is manufactured from a material that is impermeable to air, e.g. plastics or rubber. Attachable to the mask 20 are at least two conduits or tubes 30. The tubes 30 provide a means of allowing a gas to flow towards and into the mask 20, and allowing the gas to flow out of and away from the mask 20. The respective tubes 30 are open ended and a respective end of each tube 30 is attachable to the mask 20 at or adjacent a respective aperture 22. Preferably the tubes 30 are manufactured from a material that is impermeable to gas, e.g. plastics, and the attachment of a respective end of each tube 30 at or adjacent the aperture 22 is substantially airtight.

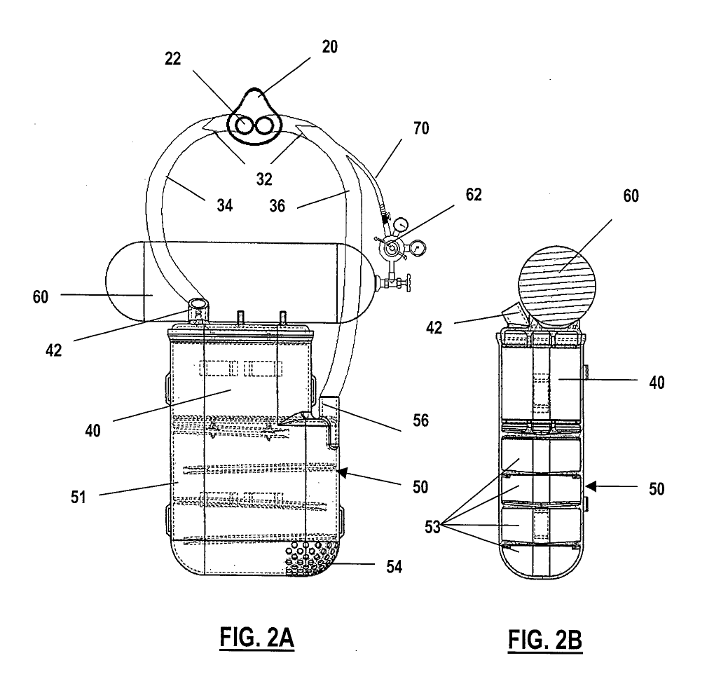

The respective tubes 30 are each provided with at least one one-way valve 32. Each one-way valve 32 provides means for allowing gas flow in one direction only through a respective tube 30, while limiting, or substantially preventing, gas flow in an opposite direction. In a preferred arrangement, shown in FIGS. 1A, 1B and 2A, a respective one-way valve 32 is located in each tube 30 adjacent the mask 20. One of the tubes 30 has its one-way valve 32 arranged so that, in use, gas may flow into the mask 20. This tube 30 is hereafter called the inhalation tube 36. The other of the tubes 30 has its one-way valve 32 arranged so that, in use, gas may flow out of the mask 20. This tube 30 is hereafter called the exhalation tube 34.

The end of the exhalation tube 34 that is distal the mask 20, is connectable to a chamber 40 at an inlet 42. Typically the chamber 40 is manufactured from gas impermeable material, e.g. plastics, and the attachment of the exhalation tube 34 to the inlet 42 is substantially airtight. The chamber 40 is provided with means for absorbing carbon dioxide from a gas and so serves in use as a carbon dioxide absorber. A variety of conventional means for absorbing carbon dioxide may be used, for example lithium hydroxide, sodium hydroxide or calcium hydroxide. The chamber 40 may also comprise a tap (not shown) for draining water resulting from the action of the carbon dioxide absorber and condensation from the user's exhaled breath.

The chamber 40 is in fluid communication with a reservoir 50 such that gas, having passed through the carbon dioxide absorber 40, travels into the reservoir 50. In the embodiment shown in FIGS. 1A and 1B, an inlet or passageway 52 is provided between the chamber 40 and the reservoir 50. Preferably, the inlet 52 is disposed substantially oppositely to the inlet 42 so that gas entering the reservoir 50 from the chamber 40 has a greater exposure to the carbon dioxide absorber than may otherwise be the case.

In the illustrated embodiment, the carbon dioxide absorber 40 is provided in the exhalation side of the breathing circuit. The carbon dioxide absorber 40 may alternatively be located on the inhalation side of the circuit, i.e. between the reservoir 50 and the mask 20.

The reservoir 50 provides means for storing gas, including gas that has been exhaled by the user and, as is described in more detail below, gas drawn from an external environment. In preferred embodiments, the reservoir 50 comprises a substantially rigid or semi rigid structure. It is preferred that the reservoir 50 is dimensioned to hold a volume of gas that is at least equal to and preferably greater than the tidal volume (inhalation or exhalation volume) of a typical user, preferably at least twice the tidal volume. The reservoir 50 is open to the external environment to allow fluid communication between the reservoir and the external environment. To this end, in the embodiment shown in FIGS. 1 and 2, a fluid port 54, or inlet/outlet, is provided in the reservoir. The port 54 may for example take the form of one or more apertures formed in the tube 51, and/or may be provided by forming part of the reservoir 50 from material that allows diffusion of gas to and from the external environment or atmosphere, e.g. a gas permeable material. Typically, the reservoir port 54 is located at an end of the reservoir 50 which is substantially opposite the inlet 52, or is at least spaced-apart form the inlet 52 so that the reservoir 50 defines a sufficiently large volume therebetween (e.g. a volume that is at least equal to the user's tidal volume).

In the embodiments shown in FIGS. 1 and 2, the reservoir 50 comprises a sinuating vessel or tube 51 having at least two alternating limb portions 53 defining an internal sinuating passageway. The inlet 52 into the reservoir 50 is located at one end of the tube 51 while the port 54 is located at the opposite end of the tube 51. The tube 51 comprises a self-supporting structure so that it does not collapse, e.g. under its own weight, at least during normal use. The tube 51 may for example be formed partially or wholly from a rigid or semi rigid material, e.g. plastics, or may comprise a frame (not shown) supporting a material which, itself, need not necessarily be rigid or semi rigid. Preferably, adjacent limbs 53 are fixed together to lend stability to the reservoir structure. Gas at one end of the tube 51, for example near the inlet 52, undergoes limited mixing with gas at the other end 54 during use. The extent to which this occurs depends on, amongst other things, the length of the tube 51 and to some extent on its shape.

The end of the inhalation tube 36 distal the mask 20 is connectable to an outlet 56 of the reservoir 50, advantageously in a substantially airtight manner. The outlet 56 is preferably located in close proximity to, or substantially adjacent, the inlet 52.

The breathing apparatus 10 also comprises, or is connectable to, at least one gas source in the form of, for example, a gas storage cylinder 60, typically storing a breathable gas, typically oxygen, held under pressure. The storage cylinder 60 provides a releasable store of breathable gas. The storage cylinder 60 preferably comprises a regulator 62 for controlling the amount of gas released. The gas is released from the regulator 62 by means of a nozzle 64. One end of a tube 70 is attachable to the nozzle 64 while the other end of the tube 70 is connectable directly or indirectly to the mask 20. For example, the other end of the tube 70 may be connected to the inhalation tube 36 or alternatively to the mask 20 at or adjacent aperture 22. The tube 70 allows gas, released from the storage cylinder 60, to enter to inhalation tube 36 and/or compartment of the mask 20. The tube 70 and the attachment of the tube 70 to the nozzle 64 and inhalation tube 36 and/or mask 20 is substantially airtight.

In alternative embodiments (not illustrated) the oxygen, or other breathable gas need not necessarily be obtained from a pressurised vessel. For example, the oxygen, or other breathable gas, may be stored chemically or in liquid form, or the apparatus may be connectable to a piped source of the gas. In general, the apparatus 10 includes or is connectable to a vessel or container containing a source of breathable gas, typically oxygen or at least including oxygen. In typical embodiments, the level of oxygen provided by the gas source is greater than that which is available from the atmosphere. Preferably, the gas source provides substantially pure oxygen, i.e. approximately 100% oxygen with substantially no contaminants.

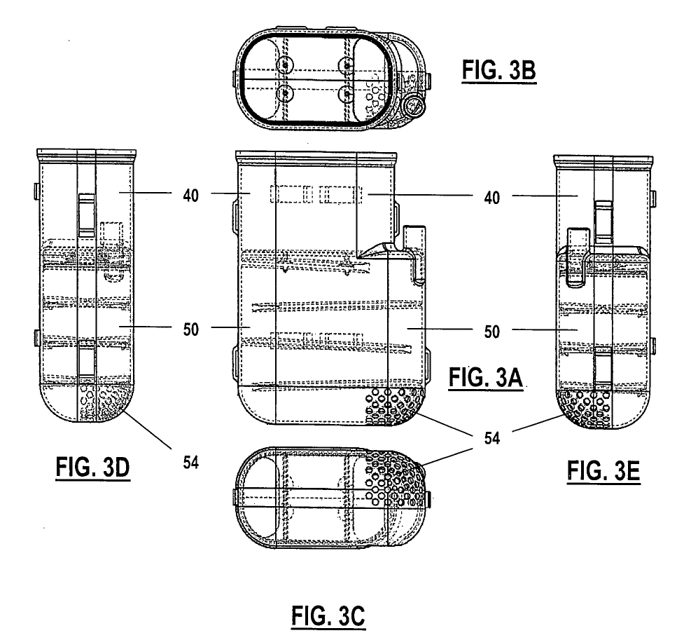

The breathing apparatus 10 may be assembled as shown in FIGS. 1A and 1B, where the chamber 40 and reservoir 50 form a compact unit and the storage cylinder 60 is attachable to the unit. An alternative embodiment of the unit is shown in FIGS. 2 and 3 where the exterior surface of the reservoir 50 and chamber 40 is smooth. In the embodiments shown in FIGS. 1 and 2, the cylinder 60 is retainable on the in-use top of the unit using securing blocks 44, conveniently in the form of wedges or chocks.

A user (not shown) may carry the apparatus 10 on their back. Fastenable shoulder straps (not shown) may be provided for this purpose. The inhalation and exhalation tubes 36, 34 may then pass over the user's shoulders so that the mask 20 may be positioned around the user's mouth and/or nose. The mask 20 may be provided with a releasably fastenable strap (not shown), which fits around the user's head to secure the mask 20 to the user's mouth and/or nose.

During use, when the user inhales, the inhalation draws gas into the mask 20 via the inhalation tube 36, the force of the inhalation causing the one-way valve 32 to open. During inhalation, gas is substantially prevented from flowing through the exhalation tube 34 into the mask 20 by the valve 32 in the exhalation tube 34.

Normally, during inhalation, the regulator 62 is operated to release a quantity of gas from the storage cylinder 60. In a preferred embodiment the gas stored and released from the storage cylinder 60 is, or at least includes, oxygen. The oxygen released from the regulator 60 is caused to flow through the tube 70 into the inhalation tube 36 and/or mask 20.

During inhalation, a quantity of gas is drawn into the mask 20 from the reservoir 50 via the outlet 56. When gas is withdrawn from the reservoir 50, it is the gas that is located proximal the outlet 56 that is withdrawn. To replace the amount of gas that is removed from the reservoir 50 via the outlet 56, an amount of gas enters the reservoir 50 from the external environment or atmosphere via the port 54. The amount of gas that enters the reservoir 50 from the atmosphere is substantially equal to the amount that was withdrawn through the outlet 56. Hence, the gas within the reservoir 50, and in particular in the portion of the reservoir 50 proximal the port 54, typically comprises atmospheric gases such as: nitrogen, carbon dioxide, oxygen and trace gases.

When the user exhales, the one-way valve 32 of the exhalation tube 34 opens to allow exhaled gas to flow through the exhalation tube 34. The exhaled gas flows into the chamber 40 via the inlet 42. The carbon dioxide absorber reduces the carbon dioxide content of gas within the chamber 40. During exhalation the one-way valve 32 of the inhalation tube 36 is closed so that gas is substantially prevented from flowing through the inhalation tube 34 from the mask 20.

Under the force of the exhalation, gas within the chamber 40 flows into the reservoir 50 via the inlet 52. The gas entering the reservoir 50 creates a displacement of gas in the tube 51 whereby gas moves substantially away from the inlet 52 and in a direction substantially towards the port 54. This causes a corresponding quantity of gas to be displaced from the reservoir via the port 54. During this action, gas is substantially prevented from leaving the reservoir 50 via outlet 56, in this embodiment by the valve 32 in the inhalation tube 36.

During the inhalation and exhalation cycles, exhaled gas that is introduced into the reservoir 50 via inlet 52 tends to remain in a region of the reservoir close to the inlet 52 and outlet 56, while gas that is drawn into to the reservoir 50 via the opening 54 tends to remain in a region of the reservoir close to the opening 54. However, some mixing of the exhaled and inducted gases does tend to occur over time and this allows the exhaled gas to be replenished with atmospheric gases. In particular, diffusion of atmospheric gases, especially nitrogen, into the breathing circuit occurs. The performance of the reservoir 50 depends on its dimensions, particularly its volume, and on the user's tidal volume. Preferably, the volume of the reservoir 50 is at least 5 litres, more preferably at least 10 litres, and most preferably approximately 12 litres.

During inhalation, the gas that is withdrawn from the reservoir 50 comprises the gas located close to the outlet 56. Due to the structure of the reservoir 50, and the limited mixing of gas within the reservoir 50, the gas that is withdrawn from the reservoir 50 during inhalation is mainly comprised of gas that entered the reservoir during exhalation. The exhaled gas is therefore recycled or “rebreathed”. By recycling or rebreathing the exhaled gas, any unused oxygen is re-inhaled, and this limits oxygen wastage. Additionally, the re-breathed gases may be heated as a result of the first inhalation thus the gas supply is partially heated which may benefit the user.

In one mode of use, the level of oxygen released from the storage cylinder 60 via the regulator 62 may be set for a desired quantity per inhalation. When the set amount of oxygen released from the storage cylinder 60 is mixed with gases drawn from the reservoir 50, the total oxygen level is dependent on the oxygen content of the gas drawn from the reservoir 50. The total oxygen content will vary if the level of oxygen in the reservoir 50 is variable.

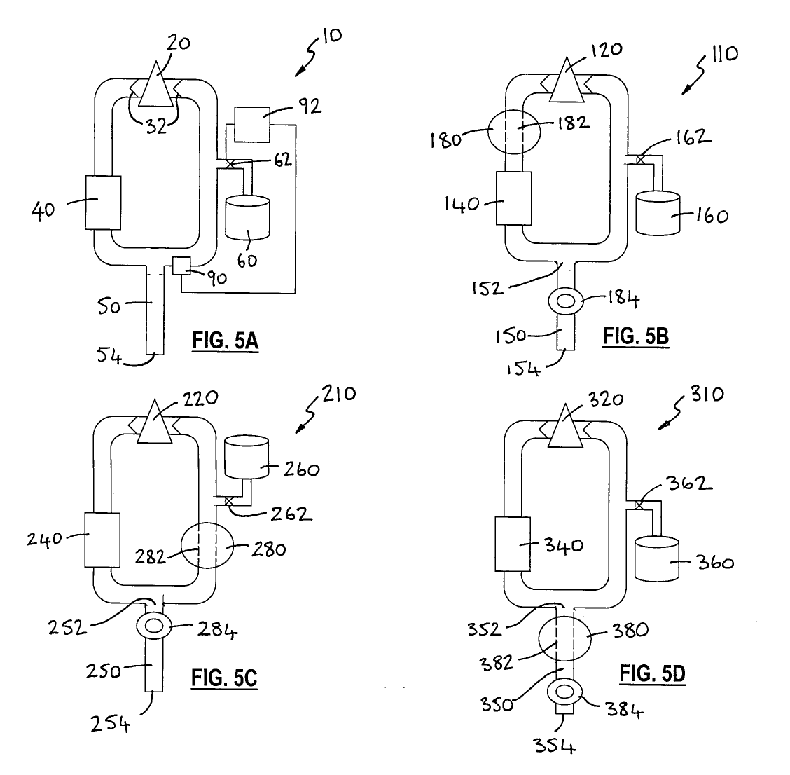

In a preferred mode of use, the level of oxygen in the breathing circuit is measurable by an oxygen monitor 90 (shown in FIG. 5A only), e.g. an oxygen sensor located in for example the reservoir 50, preferably at or adjacent the outlet 56, or located in the inhalation tube, preferably at or adjacent the outlet 56. By measuring the oxygen level in the circuit, the amount of oxygen released from the storage cylinder 60 can be controlled accordingly. This may be achieved in any suitable manner, for example by providing a control module 92 (shown in FIG. 5A only), typically comprising suitable electronic circuitry, linked to the sensor 90 and to the regulator 62 and arranged to control the operation of the regulator 62 in accordance with the level of oxygen detected by the sensor 90. The sensor 90 and control module 92 may communicate in any convenient manner, e.g. using a wired or wireless link. If the amount of oxygen in the breathing circuit decreases, then the amount of oxygen released from the storage cylinder 60 may be increased. If the amount of oxygen in circuit increases then the amount of oxygen released from the storage cylinder 60 may be decreased. Thus a substantially constant level of oxygen may be provided for inhalation.

The apparatus 10 may also be used to provide a gradually varying level of oxygen for inhalation. If the level of oxygen is desired to increase then the amount of oxygen released from the cylinder 60 may be gradually increased. In this circumstance most of the inhaled gas may be oxygen from the storage cylinder 60 and a reduced amount of inhaled gas is from the reservoir 50. If the level of oxygen is desired to decrease then the amount of oxygen released from the cylinder 60 may be decreased. In this circumstance most of the inhaled gas may comprise the gas from the reservoir 50. In conventional closed circuits, the gas that replaces oxygen when decreasing the oxygen level is provided by additional storage cylinders. However, in the apparatus 10 the replacement gases are provided from the surrounding atmosphere via the opening 54 in the reservoir 50. This is particularly advantageous when lowering the oxygen content and increasing the nitrogen content of the inhalation gas.

More generally, adjusting the amount of gas that is drawn from the cylinder 60 during inhalation causes the amount of gas that is drawn from the reservoir 50 during inhalation to be correspondingly adjusted and this, in turn, affects the extent to which exhaled gas is mixed with inducted gas in the reservoir 50 and so affects the composition of the gas drawn from the reservoir 50.

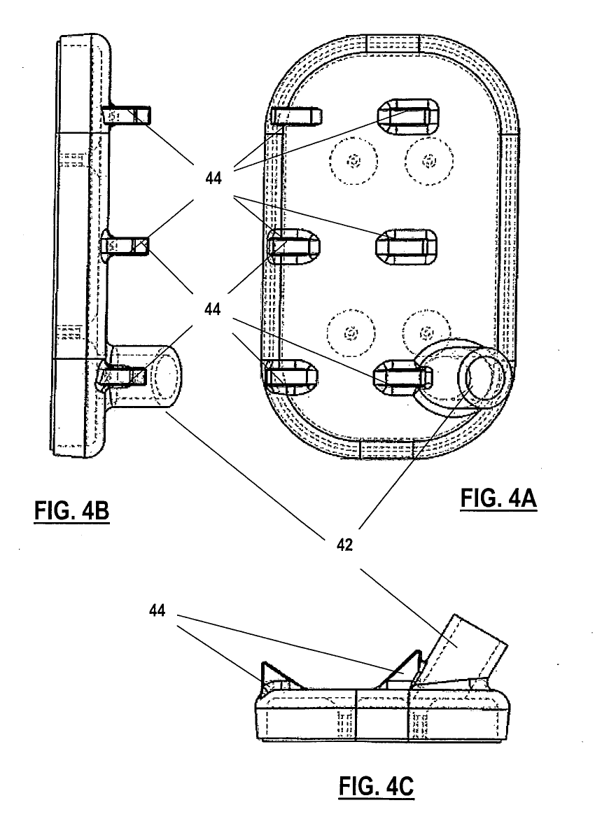

Referring now to FIGS. 5A to 5D, there are shown schematic views of breathing circuits suitable for use in apparatus embodying the invention. Like numerals are used to denote like parts. The circuit 10 of FIG. 5A corresponds to the circuit described above wither reference to FIGS. 1 to 4. The circuits 110, 210, 310 each include a collapsible or inflatable chamber 180, 280, 380 formed from a flexible material, e.g. plastics or rubber, so that it may expand and contract as the user exhales and inhales. The inflatable chamber 180, 280, 380 is fitted over or around a substantially rigid or semi-rigid portion 182, 282, 382 of the breathing circuit so that when the chamber 180, 280, 380 is in its collapsed state, the flow of air through the circuit is not prevented by the collapsed chamber, i.e. the portion 182, 282, 382 of the circuit maintains a passageway through the chamber even when it is collapsed. One or more apertures are formed in the portion 182, 282, 382 to allow gas to flow between the chamber 180, 280, 380 and the breathing circuit. Alternatively, part of the portion 182, 282, 382 may be formed from a gas permeable material to allow gas to pass into and out of the chamber 180, 280, 380.

In the circuit 110 of FIG. 5B, the inflatable chamber 180 is provided around the exhalation tube 134, between the mask 120 and the carbon dioxide absorber 140. In the circuit 210 of FIG. 5C, the inflatable chamber 280 is provided around the inhalation tube 136. In the circuit 310 of FIG. 5D, the inflatable chamber 380 is provided around a portion of the reservoir 350.

The inflatable chamber 180, 280, 380, provides additional volume to supplement that provided by the reservoir 150, 150, 350 and reduces the size of reservoir that is required.

The circuits 110, 210, 310 may optionally include a restricting or resistor device 184, 284, 384 for restricting the flow of gas in the circuit. The purpose of the resistor device 184, 284, 384 is to bias the flow of gas through the circuit such that, during exhalation, gas is caused or encouraged to fill, or at least enter, the inflatable chamber 180, 280, 380 before travelling into or through the reservoir 150, 250, 350. To this end, the resistor device 184, 284, 384 is located between the chamber 180, 280, 380 and the reservoir 150, 250, 350, or at least between the chamber 180, 280, 380 and the port 154, 254, 354 of the reservoir. The resistor device is preferably located at or adjacent the inlet 152, 252, 352 to the reservoir, or, when the chamber 180, 280, 380 is incorporated into the reservoir, between the chamber 180, 280, 380 and the port of the reservoir, preferably adjacent the outlet of the chamber 180, 280, 380.

The resistor device may take any suitable form, for example a narrowing in the respective tube 30, 51, or an insert which restricts the flow of gas.

It will be apparent from the foregoing description that the apparatus 10, 110, 210, 310 provides a breathing circuit (comprising the inhalation and exhalation tubes, the absorber and the reservoir) which allows rebreathing of exhaled gas. The circuit may be described as semi-open since it allows a limited quantity of gas to be drawn into the circuit from the external environment, while still allowing exhaled gas to be re-used. Because the reservoir is open, it is not required to expand and collapse during the inhalation/exhalation cycle and allows gas from the external environment to be introduced into the circuit without the need for a separate supply container.

It is preferable that the diameter or cross sectional area of the reservoir (and preferably also the tubes 30) is as large as possible in order to reduce resistance to gas flow and encourage gas to flow with a laminar flow rather than a turbulent flow. A larger diameter may also reduce the likelihood of the reservoir 50/tubes 30 freezing when used at low temperatures. In a preferred embodiment the reservoir and/or tubes 30 have a diameter or width of approximately 35 mm. When the apparatus 10, 110, 210, 310 is used solely at high altitudes however, the reservoir 50 and/or tubes 30 have a preferred diameter of approximately 11 mm.

In preferred embodiments, the apparatus 10, 110, 210, 310 may comprise electronic monitoring and control systems to monitor for example, the level of oxygen in the reservoir 50 and cause corresponding automatic adjustment, typically using the regulator 62, of oxygen released from the storage cylinder 60 to meet, for example, a desired oxygen content level in the inhalation gas.

In one mode of use, the apparatus 10, 110, 210, 310 acts as a supplementary oxygen device for providing the wearer with levels of oxygen that are higher than can be obtained from the atmosphere.

The preferred apparatus 10, 110, 210, 310 is operable in an open circuit mode in which the exhalation tube 34 is disconnected from the breathing circuit (e.g. disconnected from the mask 20 and/or the chamber 40). The apparatus 10, 110, 210, 310 may also comprise one or more of the following devices: a gas flow monitor, a volume monitor, one or more temperature sensors for respective locations around the circuit, an atmospheric pressure monitor, a carbon dioxide level monitor, means for calibrating the sensors and/or a GPS system. The apparatus 10, 110, 210, 310 may also comprise a data storage system so that breathing data may be stored. The carbon dioxide monitor (not shown) may be arranged to monitor the level carbon dioxide in the circuit and adjusting the flow of gas from the container 60 via regulator 62 to prevent dangerously high levels of carbon dioxide from building up.

The present invention is not limited to the embodiment(s) described herein, which may be amended or modified without departing from the scope of the present invention.

1. A breathing apparatus comprising a breathing circuit into which gas may be exhaled by a user and from which gas may be inhaled by the user, the circuit including a gas reservoir into which at least some of the exhaled gas is directed and from which at least some of the inhaled gas is drawn, wherein a portion of the gas reservoir is in fluid communication with an external environment, and wherein the circuit is connected, in use, to an apparatus for supplying breathable gas.

2. A breathing apparatus as claimed in claim 1, further including a regulating device arranged to control the quantity of gas introduced into said circuit from said apparatus for supplying breathable gas.

3. A breathing apparatus as claimed in claim 2, further including at least one gas sensor arranged to detect the level of at least one target gas in the circuit, the sensor being linked to means for controlling the operation of said regulating device in response to the detected level of said at least one target gas.

4. A breathing apparatus as claimed in claim 1, wherein said gas reservoir includes an inlet for receiving gas from the circuit, an outlet by which gas may enter the circuit from the reservoir, and a fluid port for providing said fluid communication with the external environment, said port being located distal said inlet.

5. A breathing apparatus as claimed in claim 4, wherein said outlet is located proximal said inlet.

6. A breathing apparatus as claimed in claim 1, wherein said reservoir comprises a self-supporting structure for containing said gas.

7. A breathing apparatus as claimed in claim 6, wherein said gas reservoir includes an inlet for receiving gas from the circuit, an outlet by which gas may enter the circuit from the reservoir, and a fluid port for providing said fluid communication with the external environment, said port being located distal said inlet, and wherein said reservoir structure is shaped to define a sinuating passageway between said inlet and said port.

8. A breathing apparatus as claimed in claim 1, wherein said circuit further includes an inflatable chamber arranged to expand and contract respectively as said user exhales and inhales, said chamber being incorporated into a self-supporting portion of said circuit, the arrangement being such that, when the inflatable chamber adopts a collapsed state, said self-supporting portion provides a passageway by which gas may flow through or past said inflatable chamber.

9. A breathing apparatus as claimed in claim 8, wherein said self-supporting portion of the circuit passes through said inflatable chamber, one or more apertures or gas permeable interfaces being provided in said self-supporting portion to allow gas to pass into and out of said inflatable chamber.

10. A breathing apparatus as claimed in claim 8, wherein the circuit includes a resistor device for restricting the flow of gas through the circuit, said resistor device being located between the inflatable chamber and the fluid port of the reservoir.

11. A breathing apparatus as claimed in claim 10, wherein said resistor device is located substantially at the inlet to said reservoir.

12. A breathing apparatus as claimed in claim 1, wherein the reservoir is dimensioned to define a gas-receiving volume of at least 5 litres, preferably at least 10 litres, and most preferably approximately 12 litres.

13. A breathing apparatus as claimed in claim 1, further including means for removing gas, including carbon dioxide, from the exhaled gas.

14. A breathing apparatus as claimed in claim 13, wherein the absorbing means is located in said breathing circuit such that said exhaled gas passes through said absorbing means before entering said reservoir.

15. A breathing apparatus as claimed in claim 1, wherein said apparatus for supplying a breathable gas comprises a container containing a source of said breathable gas.

16. A breathing apparatus as claimed in claim 15, wherein said container is mountable on said apparatus.

17. A breathing apparatus as claimed in claim 1, wherein said breathable gas comprises oxygen.

18. A breathing apparatus as claimed in claim 3, wherein said at least one gas sensor includes an oxygen sensor and said at least one target gas includes oxygen.

19. A breathing apparatus as claimed in claim 3, wherein said at least one gas sensor includes a carbon dioxide sensor and said at least one target gas includes carbon dioxide.
